(19) United States
(12) Patent Application Publication
(10) Pub. No.: US 2007/0106337 A1
(43) Pub. Date: May 10, 2007

(54) Methods And Apparatus For Treating Disorders Through Neurological And/Or Muscular Intervention

(75) Inventors: Joseph P. Errico, Green Brook, NJ (US); James R. Pastena, Succasunna, NJ (US); Steven Mendez, Chester, NJ (US); Hecheng Hu, Cedar Grove, NJ (US); Arthur Ross, Mendham, NJ (US)

Correspondence Address: KAPLAN GILMAN GIBSON & DERNIER L.L.P., 900 ROUTE 9 NORTH, WOODBRIDGE, NJ, 07095, US

(73) Assignee: ElectroCore, Inc., Summit, NJ (US)

(21) Appl. No.: 11/555,142

(22) Filed: Oct. 31, 2006

Related U.S. Application Data
(60) Provisional application No. 60/736,001, filed on Nov. 10, 2005.

(51) Int. Cl. A61N 1/36

(52) U.S. Cl. 607/40; 607/2

(57) ABSTRACT

Methods and apparatus proved for: simultaneously monitoring at least one of nerve and muscle electrical activity on respective sides of a target plexus of a patient; identifying desired electrical activity of at least one of nerves and muscles on both sides of the target plexus based on the monitored activity; and modulating the electrical activity of the at least one of nerves and muscles on both sides of the target plexus to achieve a therapeutic result.

### CROSS REFERENCE TO RELATED APPLICATIONS

This application claims the benefit of U.S. Provisional Patent Application No: 60/736,001, filed Nov. 10, 2005, entitled METHODS AND APPARATUS FOR TREATING DISORDERS THROUGH NEUROLOGICAL AND/OR MUSCULAR INTERVENTION, the entire disclosure of which is hereby incorporated by reference.

### BACKGROUND OF THE INVENTION

The present invention relates to methods and apparatus for treating disorders through neurological and/or muscular intervention, such as electrical modulation of one or more nerves and/or muscles.

There are a number of treatments for various infirmities that require the destruction of otherwise healthy tissue in order to affect a beneficial effect. For example, a treatment for morbid obesity that has been performed with alarming frequency is a gastric bypass, by which procedure a healthy portion of the patient's stomach is removed in order to reduce the patient's capacity to intake calories, and thus to affect the loss of significant percentages of body weight. Cholecystectomy, in some instances, is also such a surgical treatment that requires the sacrifice of an organ to alleviate pain and/or blockage of the bile duct. There are also a number of treatments of pathologies wherein malfunctioning tissue is identified, and then lesioned or otherwise compromised in order to affect a beneficial outcome, rather than attempting to repair the tissue to its normal functionality. An example of this might be the sacrifice of the Sphincter of Oddi or the burning of portions of the lower esophageal sphincter to relieve hypertension in the bile duct or prevent reflux of gastric fluids and contents in to the esophagus, respectively. While there are a variety of different techniques and mechanisms that have been designed to focus lesioning directly onto the target nerve tissue, collateral damage is inevitable.

Still other treatments for malfunctioning tissue can be medicinal in nature, in many cases leaving patients to become dependent upon artificially synthesized chemicals. Examples of this are anti-asthma drugs such as albuterol, proton pump inhibitors such as omeprazole (Prilosec), spastic bladder relievers such as Ditropan, and cholesterol reducing drugs like Lipitor and Zocor. In many cases, these medicinal approaches have side effects that are either unknown or quite significant, for example, at least one popular diet pill of the late 1990's was subsequently found to cause heart attacks and strokes.

Unfortunately, the beneficial outcomes of surgery and medicines are, therefore, often realized at the cost of function of other tissues, or risks of side effects. Fortunately, it has been recognized that electrical stimulation of the brain and/or the peripheral nervous system and/or direct stimulation of the malfunctioning tissue, which stimulation is generally a wholly reversible and non-destructive treatment, holds significant promise for the treatment of many ailments.

Electrical stimulation of the brain with implanted electrodes has been approved for use in the treatment of various conditions, including pain and movement disorders including essential tremor and Parkinson's disease. The principle behind these approaches involves disruption and modulation of hyperactive neuronal circuit transmission at specific sites in the brain. As compared with the very dangerous lesioning procedures in which the portions of the brain that are behaving pathologically are physically destroyed, electrical stimulation is achieved by implanting electrodes at these sites to, first sense aberrant electrical signals and then to send electrical pulses to locally disrupt the pathological neuronal transmission, driving it back into the normal range of activity. These electrical stimulation procedures, while invasive, are generally conducted with the patient conscious and a participant in the surgery.

Brain stimulation, and deep brain stimulation in particular, is not without some drawbacks. The procedure requires penetrating the skull, and inserting an electrode into the brain matter using a catheter-shaped lead, or the like. While monitoring the patient's condition (such as tremor activity, etc.), the position of the electrode is adjusted to achieve significant therapeutic potential. Next, adjustments are made to the electrical stimulus signals, such as frequency, periodicity, voltage, current, etc., again to achieve therapeutic results. The electrode is then permanently implanted and wires are directed from the electrode to the site of a surgically implanted pacemaker. The pacemaker provides the electrical stimulus signals to the electrode to maintain the therapeutic effect. While the therapeutic results of deep brain stimulation are promising, there are significant complications that arise from the implantation procedure, including stroke induced by damage to surrounding tissues and the neurovasculature.

An alternative to brain stimulation is to apply the electrical signal to the peripheral nerve and/or tissue directly. Obviously this is only a potentially effective treatment if and when the pathology is somehow related to this tissue, or the tissue being stimulated is in some way connected to the ailment. An example of the latter can be seen in the recently FDA approved treatment of depression refractory to medicinal treatment which is the application of electrical stimulation to the vagus nerve (also known as the tenth cranial nerve). An example of the former is disclosed in U.S. Pat. No. 6,853,862, which is directed to the use of a neurostimulator configured to apply a stimulation signal to a patient's digestive system to influence pancreatic exocrine and endocrine secretions. In this case, it is understood that the brain controls the functioning of the stomach and the pancreas through signals that travel between it and the organs in part along the vagus nerve. In this case, a stimulation signal is applied to a patient's digestive system via at least one electrical lead positioned in the patient's abdomen, where the stimulation signal is adapted to influence pancreatic exocrine secretions or to influence pancreatic endocrine secretions. The '862 patent discloses that electrical stimulation of the vagus nerve or digestive system causes impulses that may result in pancreatic stimulation. The patent goes on to state that impulses caused by electrical stimulation of the vagus nerve or digestive system can travel by means of both afferent and efferent pathways to the pancreas. The '862 patent states that some impulses can travel from the digestive system, along a vagal afferent pathway to the brain, and then along an efferent pathway from the brain to the pancreas.

While the inventors do not have data specific regarding the results obtained using the method disclosed in the '862 patent, in general the success of treating digestive and other disorders using electrical modulation of nerves, such as the vagus nerve or musculature, is not satisfactory. Indeed, only about 20% of patients achieve significant therapeutic results.

Although the present invention is not limited to any particular theory of operation, it is believed that the pancreatic dysfunction is related to a failure of either the brain to send the proper signal to the pancreas, or that the proper signal, while generated in the brain is not being transmitted to the pancreas properly along the vagus nerve. If the problem with the pancreas is internal to the organ itself (i.e., genetic malformation, infection, etc.) this treatment will be of limited effectiveness.

It is important to note, however, that the digestive system, and the pancreas specifically, are innervated by nerves other than the vagus nerve as well. The sympathetic nerve chain, and to the extent that the spinal nerve roots are incorporated with the sympathetic fibers extending out from the sympathetic ganglia, also innervate the organs of the thoracic and abdominal cavities. Therefore, if the pathological dysfunction of the pancreas, for example, is related to a failure of a regulatory signal to be transmitted to the organ through the fibers of the sympathetic chain, stimulation of the vagus nerve may have little effect.

This is not to suggest that simply stimulating the sympathetic nerve fibers, and foregoing any attention of the vagus nerve is a superior treatment, as is suggested in U.S. Pat. No. 6,609,030 to Rezai.

Accordingly, there are needs in the art for new methods and apparatuses for treating disorders through neurological and/or other tissue stimulation means that take into consideration the more complex innervation of organs than simply by a single nerve (such as the vagus nerve).

It is of consequence to applications of the present invention to note that the '862 patent identifies the stomach as a location in the digestive system well suited for stimulation of the vagus nerve because the wall of the stomach is well enervated by the vagus nerve.

Similarly, it is of note that the '030 patent shows the viability of approaching the sympathetic fibers for electrical stimulation.

### SUMMARY OF THE INVENTION

The inventors of the present invention submit that the cause of many physiological disorders may be a dysfunction in any one nerve, or a combination of nerves and/or nerve clusters, known as ganglia and/or plexuses, and that the proper treatment of such a dysfunction by electrical stimulation cannot be effective without a method that takes these alternative pathologies into consideration. More particularly, with respect to organ function, including but not limited to the respiratory, cardiovascular, digestive, reproductive, and renal-urinary systems, the nerves most directly involved with motor and sensory control are those of the tenth cranial nerve (the vagus nerve) and the sympathetic nerves. It shall be understood that the sympathetic nerve fibers emanating from the chain that extends along the anterior outside of the vertebral column, in conjunction with the fibers of the spinal cord nerve roots that join with the sympathetic fibers, form the sympathetic nervous system. The plexuses and ganglia, such as the celiac, pulmonary, cardiac, hepatic, mesenteric plexuses, that control the organ function are formed, from one side by, the afferent and efferent fibers of the vagus nerve (or in limited instances by others of the cranial nerves) and on the other side by the fibers of the sympathetic nervous system. The present invention has applicability in treating disorders that benefit from simultaneous monitoring and/or modulation of one or more sympathetic nerves, or one or more cranial nerves, or the plexus formed by the interaction of the two.

Specifically, the treatment regiments contemplated by the inventors of the present invention include the holistic monitoring of at least two of (i) the sympathetic nerve fibers (at a location distal to the sympathetic chain such that the spinal cord nerve root fibers are incorporated into the fiber bundle), (ii) the fibers of the cranial nerve branch responsible for communication with the organ or target tissue, (iii) the plexus wherein these two nerve fibers communicate, (iv) the muscles surrounding or interfacing with the pathologically responding tissue, and (v) any physical state of being that may be associated with the condition, and thusly creating a stimulation signal pattern based upon the evaluation of the monitoring such that the desired therapeutic effect results.

More specifically, the inventors hereof have made the realization that the control of the organ and/or tissue is the result of a circuit that begins in the brain, and may include at least three separate descending components, i.e., the cranial nerve, the sympathetic nerve fibers, and the spinal cord nerve roots. This circuit is, in fact, an electrical circuit, and most importantly it is being disclosed herein that it is most effective, when attempting to modify the behavior of a component in an electrical circuit, to determine the nature and function of as many of (and preferably all of) the components of the circuit before simply driving a signal into the system. This requires monitoring the appropriate components and accurately analyzing the results of that monitoring.

Physiological disorders that may be treated by this monitoring of the entire circuit, and then applying the corrective signal to the appropriate component of the system, include, but are not limited to intestinal motility disorders, sexual dysfunction, bronchial disorder (such as asthma), dysfunction of the liver, pancreatic disorders, and heart disorders, pulmonary disorders, gastrointestinal disorders, and renal and urinary complaints. The number of disorders to be treated is limited only by the number, variety, and placement of electrodes (or combinations of multiple electrodes) along the sympathetic nervous system and cranial nervous system.

An example might be the treatment of obesity, and more particularly, obesity that results from overeating as a consequence of the patient's failure to produce a normal satiety impulse. In this circumstance, the inventors hereof disclose that it may be effective to monitor the activity in (i) the greater and/or lesser splanchnic nerves, (ii) the left and/or right branches of the vagus nerve, (iii) the celiac plexus, (iv) the pyloric valve, and (v) the muscle activity in the stomach wall. It may be found that the celiac plexus fails to activate when the muscles of the stomach distend and begin the digestive cycle, despite the fact that the vagal nerve fibers are carrying a sufficient activation signal. In such an instance, a signal applied to the sympathetic nerve fibers may be appropriate. Alternatively, it may be found that the feeling of satiety may ultimately be realized late by the patient, but only after significant overeating has already occurred, in which case the application of the indigenous neural impulses associated with satiety earlier during eating may be appropriate

Ultimately, the inventors hereof recognize that the treatment of disorders having common symptoms may have entirely different causes, and as such must be distinguished from one another if an effective treatment is to be developed. Nowhere is this principle truer than in the potential treatment of ailments through stimulation of the nerves that control the peripheral organs and/or tissues.

Other aspects, features, and advantages of the present invention will be apparent to one skilled in the art from the description herein taken in conjunction with the accompanying drawings.

### DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 is a schematic diagram of the human autonomic nervous system, illustrating sympathetic fibers, spinal nerve root fibers, and cranial nerves;

FIG. 2 is a further schematic diagram of the human autonomic nervous system and a modulation system therefore in accordance with one or more embodiments of the present invention;

FIG. 3 is a process flow diagram illustrating process steps that may be carried out for the treatment of disorders using neuromuscular modulation in accordance with one or more embodiments of the present invention; and

FIG. 4 is a graphical illustration of an electrical signal profile that may be used to treat disorders through neuromuscular modulation in accordance with one or more embodiments of the present invention.

### DETAILED DESCRIPTION OF THE PREFERRED EMBODIMENTS

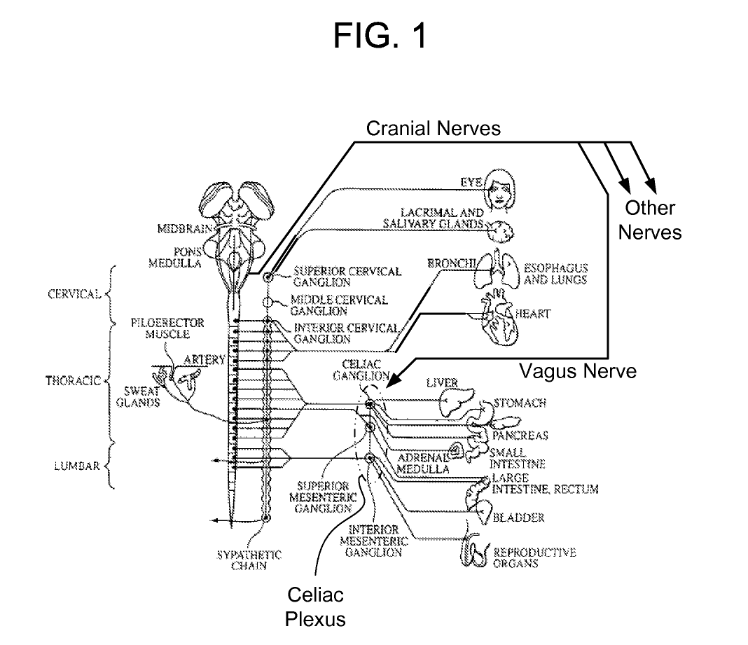

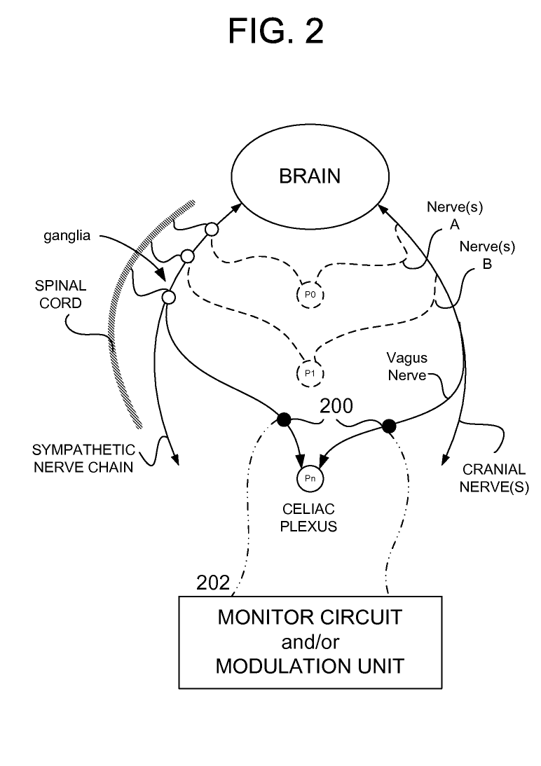

With reference to the drawings wherein like numerals indicate like elements there are shown in FIGS. 1 and 2 schematic diagrams of the human autonomic nervous system, including sympathetic fibers, parasympathetic fibers, and cerebral nerves.

The sympathetic nerve fibers, along with many of the spinal cord's nerve root fibers, and the cranial nerves that innervate tissue in the thoracic and abdominal cavities are sometimes referred to as the autonomic, or vegetative, nervous system. The sympathetic, spinal, and cranial nerves all have couplings to the central nervous system, generally in the primitive regions of the brain, however, these components have direct effects over many regions of the brain, including the frontal cortex, thalamus, hypothalamus, hippocampus, and cerebellum. The central components of the spinal cord and the sympathetic nerve chain extend into the periphery of the autonomic nervous system from their cranial base to the coccyx, essentially passing down the entire spinal column, including the cervical, thoracic and lumbar regions. The sympathetic chain extends on the anterior of the column, while the spinal cord components pass through the spinal canal. The cranial nerves, the one most innervating of the rest of the body being the vagus nerve, passes through the dura mater into the neck, and then along the carotid and into the thoracic and abdominal cavities, generally following structures like the esophagus, the aorta, and the stomach wall.

Because the autonomic nervous system has both afferent and efferent components, modulation of its fibers can affect both the end organs (efferent) as well as the brain structure to which the afferents fibers are ultimately coupled within the brain.

Although sympathetic and cranial fibers (axons) transmit impulses producing a wide variety of differing effects, their component neurons are morphologically similar. They are smallish, ovoid, multipolar cells with myelinated axons and a variable number of dendrites. All the fibers form synapses in peripheral ganglia, and the unmyelinated axons of the ganglionic neurons convey impulses to the viscera, vessels and other structures innervated. Because of this arrangement, the axons of the autonomic nerve cells in the nuclei of the cranial nerves, in the thoracolumbar lateral comual cells, and in the gray matter of the sacral spinal segments are termed preganglionic sympathetic nerve fibers, while those of the ganglion cells are termed postganglionic sympathetic nerve fibers. These postganglionic sympathetic nerve fibers converge, in small nodes of nerve cells, called ganglia that lie alongside the vertebral bodies in the neck, chest, and abdomen. The effects of the ganglia as part of the autonomic system are extensive. Their effects range from the control of insulin production, cholesterol production, bile production, satiety, other digestive functions, blood pressure, vascular tone, heart rate, sweat, body heat, blood glucose levels, and sexual arousal.

The parasympathetic group lies predominately in the cranial and cervical region, while the sympathetic group lies predominantly in the lower cervical, and thoracolumbar and sacral regions. The sympathetic peripheral nervous system is comprised of the sympathetic ganglia that are ovoid/bulb like structures (bulbs) and the paravertebral sympathetic chain (cord that connects the bulbs). The sympathetic ganglia include the central ganglia and the collateral ganglia.

The central ganglia are located in the cervical portion, the thoracic portion, the lumbar portion, and the sacral portion. The cervical portion of the sympathetic system includes the superior cervical ganglion, the middle cervical ganglion, and the interior cervical ganglion.

The thoracic portion of the sympathetic system includes twelve ganglia, five upper ganglia and seven lower ganglia. The seven lower ganglia distribute filaments to the aorta, and unite to form the greater, the lesser, and the lowest splanchnic nerves. The greater splanchnic nerve (splanchnicus major) is formed by branches from the fifth to the ninth or tenth thoracic ganglia, but the fibers in the higher roots may be traced upward in the sympathetic trunk as far as the first or second thoracic ganglion. The greater splanchnic nerve descends on the bodies of the vertebrae, perforates the crus of the diaphragm, and ends in the celiac ganglion of the celiac plexus. The lesser splanchnic nerve (splanchnicus minor) is formed by filaments from the ninth and tenth, and sometimes the eleventh thoracic ganglia, and from the cord between them. The lesser splanchnic nerve pierces the diaphragm with the preceding nerve, and joins the aorticorenal ganglion. The lowest splanchnic nerve (splanchnicus imus) arises from the last thoracic ganglion, and, piercing the diaphragm, ends in the renal plexus.

The lumbar portion of the sympathetic system usually includes four lumbar ganglia, connected together by interganglionic cords. The lumbar portion is continuous above, with the thoracic portion beneath the medial lumbocostal arch, and below with the pelvic portion behind the common iliac artery. Gray rami communicantes pass from all the ganglia to the lumbar spinal nerves. The first and second, and sometimes the third, lumbar nerves send white rami communicantes to the corresponding ganglia.

The sacral portion of the sympathetic system is situated in front of the sacrum, medial to the anterior sacral foramina. The sacral portion includes four or five small sacral ganglia, connected together by interganglionic cords, and continuous above with the abdominal portion. Below, the two pelvic sympathetic trunks converge, and end on the front of the coccyx in a small ganglion.

The collateral ganglia include the three great gangliated plexuses, called, the cardiac, the celiac (solar or epigastric), and the hypogastric plexuses. The great plexuses are respectively situated in front of the vertebral column in the thoracic, abdominal, and pelvic regions. They consist of collections of nerves and ganglia; the nerves being derived from the sympathetic trunks and from the cerebrospinal nerves. They distribute branches to the viscera.

Although all of the great plexuses (and their sub-parts) are of interest in accordance with various embodiments of the present invention, by way of example, the celiac plexus is shown in FIGS. 1 and 2 in more detail. The celiac plexus is the largest of the three great sympathetic plexuses and is located at the upper part of the first lumbar vertebra. The celiac plexus is composed of the celiac ganglia and a network of nerve fibers uniting them together. The celiac plexus and the ganglia receive the greater and lesser splanchnic nerves of both sides and some filaments from the right vagus nerve. The celiac plexus gives off numerous secondary plexuses along the neighboring arteries. The upper part of each celiac ganglion is joined by the greater splanchnic nerve, while the lower part, which is segmented off and named the aorticorenal ganglion, receives the lesser splanchnic nerve and gives off the greater part of the renal plexus.

The secondary plexuses associated with the celiac plexus consist of the phrenic, hepatic, lineal, superior gastric, suprarenal, renal, spermatic, superior mesenteric, abdominal aortic, and inferior mesenteric. The phrenic plexus emanates from the upper part of the celiac ganglion and accompanies the inferior phrenic artery to the diaphragm, with some filaments passing to the suprarenal gland and branches going to the inferior vena cava, and the suprarenal and hepatic plexuses. The hepatic plexus emanates from the celiac plexus and receives filaments from the left vagus and right phrenic nerves. The hepatic plexus accompanies the hepatic artery and ramifies upon its branches those of the portal vein in the substance of the liver. Branches from hepatic plexus accompany the hepatic artery, the gastroduodenal artery, and the right gastroepiploic artery along the greater curvature of the stomach.

The lienal plexus is formed from the celiac plexus, the left celiac ganglion, and from the right vagus nerve. The lienal plexus accompanies the lienal artery to the spleen, giving off subsidiary plexuses along the various branches of the artery. The superior gastric plexus accompanies the left gastric artery along the lesser curvature of the stomach, and joins with branches from the left vagus nerve. The suprarenal plexus is formed from the celiac plexus, from the celiac ganglion, and from the phrenic and greater splanchnic nerves. The suprarenal plexus supplies the suprarenal gland. The renal plexus is formed from the celiac plexus, the aorticorenal ganglion, and the aortic plexus, and is joined by the smallest splanchnic nerve. The nerves from the suprarenal plexus accompany the branches of the renal artery into the kidney, the spermatic plexus, and the inferior vena cava.

The spermatic plexus is formed from the renal plexus and aortic plexus. The spermatic plexus accompanies the internal spermatic artery to the testis (in the male) and the ovarian plexus, the ovary, and the uterus (in the female). The superior mesenteric plexus is formed from the lower part of the celiac plexus and receives branches from the right vagus nerve.

The superior mesenteric plexus surrounds the superior mesenteric artery and accompanies it into the mesentery, the pancreas, the small intestine, and the great intestine. The abdominal aortic plexus is formed from the celiac plexus and ganglia, and the lumbar ganglia. The abdominal aortic plexus is situated upon the sides and front of the aorta, between the origins of the superior and inferior mesenteric arteries, and distributes filaments to the inferior vena cava. The inferior mesenteric plexus is formed from the aortic plexus. The inferior mesenteric plexus surrounds the inferior mesenteric artery, the descending and sigmoid parts of the colon and the rectum.

While the sympathetic and parasympathetic nervous system extends between the brain and the great plexuses, the cranial nerves extend between the brain and the great plexuses along other paths. For example, as best seen in FIG. 2, the sympathetic and parasympathetic nerves extend between the brain the celiac plexus along a first portion of a “circuit,” while the vagus nerve extends between the brain the celiac plexus along a second portion of the same circuit.

There are twelve pairs of cranial nerves, namely: the olfactory, optic, oculomotor, trochlear, trigeminal, abducent, facial, acoustic, glossopharyngeal, vagus, accessory, and hypoglossal. The nuclei of origin of the motor nerves and the nuclei of termination of the sensory nerves are brought into relationship with the cerebral cortex.

Although all of the cranial nerves are of interest in accordance with various embodiments of the present invention, by way of example, the vagus nerve is shown in FIGS. 1 and 2 in more detail. The vagus nerve is composed of motor and sensory fibers and is of considerable interest in connection with various embodiments of the present invention because it has a relatively extensive distribution than the other cranial nerves and passes through the neck and thorax to the abdomen. The vagus nerves leaves the cranium and is contained in the same sheath of dura mater with the accessory nerve. The vagus nerve passes down the neck within the carotid sheath to the root of the neck. On the right side, the nerve descends by the trachea to the back of the root of the lung, where it spreads out in the posterior pulmonary plexus. From the posterior pulmonary plexus, two cords descend on the esophagus and divide to form the esophageal plexus. The branches combine into a single cord, which runs along the back of the esophagus, enters the abdomen, and is distributed to the posteroinferior surface of the stomach, joining the left side of the celiac plexus, and sending filaments to the lienal plexus.

On the left side, the vagus nerve enters the thorax, crosses the left side of the arch of the aorta, and descends behind the root of the left lung, forming the posterior pulmonary plexus. From posterior pulmonary plexus, the vagus nerve extends along the esophagus, to the esophageal plexus, and then to the stomach. The vagus nerve branches over the anterosuperior surface of the stomach, the fundus, and the lesser curvature of the stomach.

The branches of distribution of the vagus nerve are as follows: the auricular, the superior laryngeal, the recurrent, the superior cardiac, the inferior cardiac, the anterior bronchial, the posterior bronchial, the esophageal, the celiac, and the hepatic. Although all of the branches of the vagus nerve are of interest in accordance with various embodiments of the invention, the gastric branches and the celiac branches are believed to be of notable interest. The gastric branches are distributed to the stomach, where the right vagus nerve forms the posterior gastric plexus on the postero-inferior surface of the stomach and the left vagus nerve forms the anterior gastric plexus on the antero-superior surface of the stomach. The celiac branches are mainly derived from the right vagus nerve, which enter the celiac plexus and supply branches to the pancreas, spleen, kidneys, suprarenal bodies, and intestine.

One or more embodiments of the present invention provide for one or more methods of treating physiological disorders by at least one of monitoring and modulating one or more nerves and/or one or more muscles on both sides of a particular plexus. Although the various embodiments of the invention are not limited by any particular theory of operation, it is believed that advantages are obtained when the disorder is associated with organs and/or musculature enervated by the nerves entering or leaving the given plexus. For example, it is believed that disorders associated with digestion (e.g., overeating, satiety, acid reflux, acid production, stomach activity, etc.) may be better treated through electronic monitoring and/or electro-modulation of the nerves and/or musculature on both sides of the esophageal, celiac, and hepatic plexuses. In particular, it is believed that electrical (or chemical) modulation of: (i) one or more of the sympathetic or parasympathetic nerves (discussed above) on the one side of the appropriate plexus; and (ii) one or more of the vagus nerves (also discussed above) on the other side of the appropriate plexus, will improve the therapeutic effect on one or more pathologies. For example, pathological hunger and the urge to eat beyond the point of normal satiety may best be treated by monitoring, and appropriately stimulating one or more nerves based upon the results of the monitoring the fibers of the greater and lesser splanchnic nerves, the celiac plexus, and/or the right vagus nerve fibers on the lesser curvature of the stomach.

Similarly, esophageal reflux, including GERD and other pathologies involved in the retrograde flow of gastric contents into the esophagus may best be treated by monitoring, and then appropriately stimulating based upon the results of the monitoring the neural activity in the sympathetic nerve fibers and vagus nerve fibers that innervate the esophageal plexus.

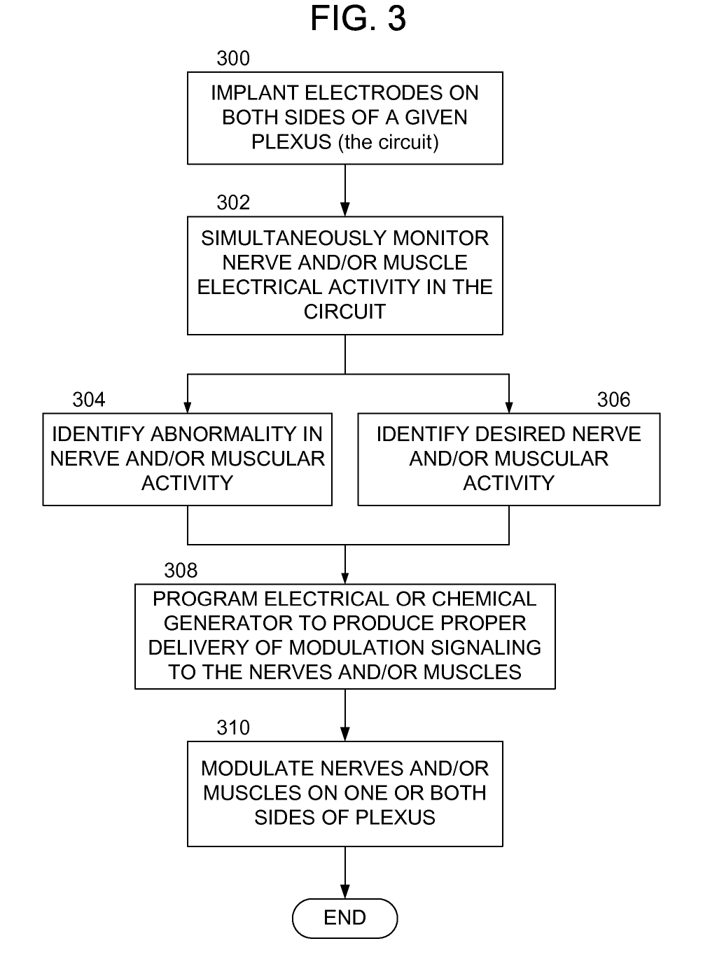

Further reference is now made to FIG. 3, which illustrates a process flow of steps or actions, one or more of which may be carried out in accordance with one or more embodiments of the present invention. At action 300, one or more electrodes 200 are implanted on or near at least one of the sympathetic or parasympathetic nerves on one side of a target plexus, such as the celiac plexus. On or more further electrodes 200 are implanted on or near at least one of the cranial nerves entering or leaving the target plexus, or on or near at least one of the muscles enervated by such nerves. The electrodes 200 may be configured as monopolar electrodes, with one electrode 200 per lead, or as multipolar electrodes, with more than one electrode 200 per lead. Preferably, the electrodes 200 are made from a biocompatible conductive material such as platinum-iridium. Any of the known electrodes and leads may be used for this purpose (such as from Medtronic, Model 4300). The electrodes 200 are attached to the electrical leads prior to implantation and navigated to a point near the desired modulation site. The electrical leads and electrodes 200 may be surgically inserted into the patient using a surgical technique, such as laparotomy or laparoscopy, with proximal ends of the leads located near the modulation unit 202 and distal ends located near the desired modulation site.

At action 300 simultaneous monitoring of the nerve and/or muscle activity on both sides of the target plexus is performed using the monitor circuit 202. Any of the known equipment operable to receive electrical signaling from the electrodes 200 and to produce graphic and/or tabular data therefrom may be employed. It is desirable that the monitor circuit 202 and/or a computer associated therewith is capable of correlating and/or analyzing the received data to identify abnormalities in the activity of the nerves and/or muscles (action 304) or to identify a desired activity of the nerves and/or muscles (action 306) to achieve the therapeutic effect. For example, if a digestive disorder (e.g., overeating) were to be treated, the measured activity of the nerves and/or muscles of the patient may indicate an abnormal satiety profile. If so, a desired satiety profile may be formulated, which if achieved through modulation of the nerves and/or muscles would result in a reduced desire to eat on the part of the patient.

At action 308, the modulation unit 202 is preferably programmed to modulate the nerves and/or muscles on one or both sides of the target plexus to achieve the therapeutic result (action 310). The modulation may be achieved through electrical and/or chemical intervention. In the case of electrical modulation, the preferred effect may be to stimulate or reversibly block nervous and or muscular tissue. Use of the term block means disruption, modulation, and/or inhibition of nerve impulse transmission and/or muscular flexion and inhibition. Abnormal regulation can result in an excitation of the pathways or a loss of inhibition of the pathways, with the net result being an increased perception or response. Therapeutic measures can be directed towards either blocking the transmission of signals or stimulating inhibitory feedback. Electrical stimulation permits such stimulation of the target neural structures and, equally importantly, prevents the total destruction of the nervous system. Additionally, electrical stimulation parameters can be adjusted so that benefits are maximized and side effects are minimized.

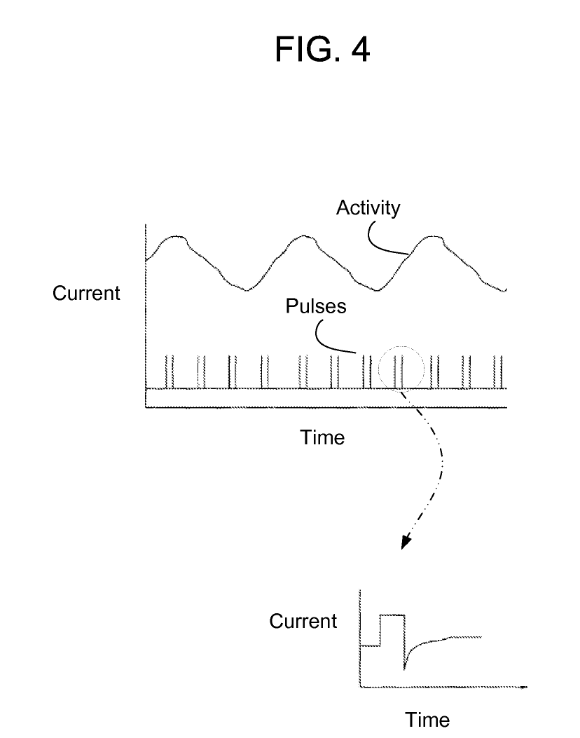

With reference to FIG. 4, the electrical voltage/current profile of the modulation signal to the electrodes 200 (and thus the nerves/muscles) may be achieved using a pulse generator. In a preferred embodiment, the modulation unit 202 includes a power source, a processor, a clock, a memory, etc. to produce a pulse train to the electrodes 200. The parameters of the modulation signal are preferably programmable (action 308), such as the frequency, amplitude, duty cycle, pulse width, pulse shape, etc. The modulation unit 202 may be surgically implanted, such as in a subcutaneous pocket of the abdomen or positioned outside the patient. By way of example, the modulation unit 202 may be purchased commercially, such as the Itrel 3 Model 7425 available from Medtronic, Inc. The modulation unit 202 is preferably programmed with a physician programmer, such as a Model 7432 also available from Medtronic, Inc.

The electrical leads and electrodes 200 are preferably selected to achieve respective impedances permitting a peak pulse current in the range from about 0.01 mA to about 100.0 mA.

The modulation signal may have a frequency selected to influence the therapeutic result, such as from about 0.2 pulses per minute to about 18,000 pulses per minute, depending on the application. The modulation signal may have a pulse width selected to influence the therapeutic result, such as from about 0.01 ms to 500.0 ms. The modulation signal may have a peak current amplitude selected to influence the therapeutic result, such as from about 0.01 mA to 100.0 mA.

In addition, or as an alternative to the devices to implement the modulation unit 202 for producing the electrical voltage/current profile of the modulation signal to the electrodes 200, the device disclosed in U.S. Patent Publication No.: 2005/0216062 (the entire disclosure of which is incorporated herein by reference) may be employed. U.S. Patent Publication No.: 2005/0216062 discloses a multi-functional electrical stimulation (ES) system adapted to yield output signals for effecting faradic, electromagnetic or other forms of electrical stimulation for a broad spectrum of different biological and biomedical applications. The system includes an ES signal stage having a selector coupled to a plurality of different signal generators, each producing a signal having a distinct shape such as a sine, a square or a saw-tooth wave, or simple or complex pulse, the parameters of which are adjustable in regard to amplitude, duration, repetition rate and other variables. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated as well as the outputs of various sensors which sense conditions prevailing in this substance whereby the user of the system can manually adjust it or have it automatically adjusted by feedback to provide an electrical stimulation signal of whatever type he wishes and the user can then observe the effect of this signal on a substance being treated.

As discussed above, the therapeutic treatment may also additionally or alternatively include using a pharmaceutical drug or drugs to modulate the nerves and/or muscles. This may be accomplished by means of an implantable pump and a catheter to administer the drug(s). The catheter preferably includes a discharge portion that lies adjacent a predetermined infusion site, e.g., one or more of the sites discussed above (or below) in the treatment. The modulation unit 202 is preferably operable to communicate with the pump to administer the drug(s) at predetermined dosage(s) in order to treat the disorder.

### PROPHETIC EXAMPLE 1

By way of example, one or more embodiments of the present invention are believed useful in treating obesity. One way of treating obesity is to modulate the satiety level of the patient, for example, under the theory that if the patient is satisfied he or she will not overeat. In this regard, it is contemplated that nerves are monitored and modulated on both sides of (and possibly at) the celiac plexus because nerves associated with the celiac plexus terminate and originate at the stomach. By monitoring and/or modulating nerves in the circuit through the celiac plexus it is believed that superior therapeutic results will be obtained.

It is believed that in the contemplated treatment the nerves of the greater and lesser splanchnic nerves are good candidates for monitoring/modulation on one side of the celiac plexus. Recall that the greater splanchnic nerve descends on the bodies of the vertebrae, perforates the crus of the diaphragm, and ends in the celiac ganglion of the celiac plexus. The lesser splanchnic nerve pierces the diaphragm and joins the aorticorenal ganglion. Nerve branches from the hepatic plexus (of the celiac plexus) accompany the hepatic artery, the gastroduodenal artery, and the right gastroepiploic artery along the greater curvature of the stomach. The superior gastric plexus accompanies the left gastric artery along the lesser curvature of the stomach, and joins with branches from the left vagus nerve.

It is believed that in the contemplated treatment the gastric and celiac branches of the vagus nerve are good candidates for monitoring/modulation on the other side of the celiac plexus. Recall that the gastric branches and the celiac branches are distributed to the stomach: the right vagus nerve forms the posterior gastric plexus on the postero-inferior surface of the stomach and the left vagus nerve forms the anterior gastric plexus on the antero-superior surface of the stomach. The celiac branches are mainly derived from the right vagus nerve, which enter the celiac plexus and supply branches to the pancreas, spleen, kidneys, suprarenal bodies, and intestine.

Once the nerves in the circuit are identified, one or more electrodes 200 are implanted on or near the nerves on both sides of the celiac plexus. Next, simultaneous monitoring of the nerves on both sides of the celiac plexus is performed using the monitor circuit 202. The data are correlated and analyzed to identify abnormalities in the activity of the nerves and to identify a desired activity of the nerves and/or muscles to achieve the therapeutic effect. The modulation unit 202 is preferably programmed to modulate the nerves on one or preferably both sides of the celiac plexus, preferably through electrical intervention.

### PROPHETIC EXAMPLE 2

By way of further example, one or more embodiments of the present invention are believed useful in treating obesity by monitoring and/or modulating muscles of the stomach and/or intestine (preferably on both sides of the celiac plexus), preferably those muscles associated with one or more of the nerves identified in EXAMPLE 1.

### PROPHETIC EXAMPLE 3

By way of further example, one or more embodiments of the present invention are believed useful in treating other digestive disorders, for example, gastric reflux. The stomach includes an opening through which the esophagus communicates therewith—known as the cardiac orifice. The orifice includes a sphincter muscle that opens to accept food and closes to prevent stomach contents (including acid) to enter the esophagus. It is believed that the nerves and/or muscles associated with acid production and/or the sphincter of the cardiac orifice may be monitored and/or modulated to prevent reflux. For example, the sphincter may be modulated to close tightly at night (when the patient is prone) to prevent stomach acids from entering the esophagus. Preferably, the modulation occurs as a result of monitoring nerve and or muscle activity on both sides of the celiac plexus, preferably those nerves/muscles associated with one or more of the nerves identified in EXAMPLE 1.

### PROPHETIC EXAMPLE 4

Further examples of one or more embodiments of the present invention are believed useful in treating other disorders associated with the nerves, muscles, hormones associated with the stomach, pancreas, spleen, kidneys, suprarenal bodies, gall bladder, etc. Indeed, using the description of the anatomy herein (or other sources) nerves and/or muscles may be identified on two sides of a target plexus highly associated with the viscera of interest. Monitoring and modulation treatment may then ensue.

### PROPHETIC EXAMPLE 5

An application for the treatment of esophageal reflux might include the monitoring of one or more of (i) the nerve fibers of the lower cervical and/or upper thoracic sympathetic nerve chain, (ii) the right and left vagus nerve branches, (iii) one or more plexus of the esophagus, (iv) the lower esophageal sphincter, and (v) the pH of the esophagus. The monitoring may show that during certain periods of low activation of the lower esophageal sphincter, when the pH is found to rise above desired levels (evidencing periods of reflux), the sympathetic nerve fibers are inactive despite strong motor signals through the esophageal plexuses. This result might suggest that an artificially applied stimulation of the sympathetic nerve fibers whenever the vagus nerve fibers are firing but the lower esophageal sphincter is not tightening. Alternatively, it might be found that the sympathetic and vagus nerves are failing to provide the appropriate signal to the lower esophageal sphincter, in which case the stimulation may be more appropriately applied directly to the muscle of the sphincter during periods wherein the pH in the esophagus rises above a predetermined amount. Still further, the vagus nerve and sympathetic nerves may be sending the appropriate signal, but it is delayed as the result of a failure of the body to recognize the rising pH levels until too high a level has already been reached. In such a circumstance, the application of the indigenous signals might be applied whenever the pH level rises, and the signal may be shut off once the indigenous neural activity begins.

### PROPHETIC EXAMPLE 6

Other treatments in accordance with embodiments of the invention may address urinary incontinence, asthma, erectile dysfunction, and bile hypertension.

### PROPHETIC EXAMPLE 7

In a further embodiment of the present invention, a treatment system may employ electrical signals to modify and/or control the digestive system of a patient, which may alternatively or additionally include controlling release of chemicals and/or hormones that influence digestion. Electrical signals may be applied directly to the digestive organs, muscles, sphincters, surrounding tissue, nerves, and/or plexuses. Chemicals and/or hormones can be stimulated from the body or released from reservoirs that are part of the treatment system.

Command(s) to the digestive system can be based on: (i) patient input (e.g., through wireless telemetry or magnet/reed switch(es)) resulting from pain sensations or meal/bed time habits, etc.; (ii) responses to sensor data such as pressure in the patient's gall bladder or duct(s), nerve signals, stomach muscle signals, concentration of enzymes and/or hormones; (iii) physician pre-programmed schedules; and/or (iv) a default software program in the stimulator.

A valve and/or stent can be used to augment and/or replace damaged or diseased sphincters, ducts, etc. The valve opens and closes with an electrical signal based on the commands described above. The stent may be flexible so that sphincter contraction would still close the opening, or the stent material itself may respond to electrical signals to change shape. The stent may also be combined with a sensor to detect chemicals or pressure/flow information. The treatment system may have a stent/valve maintenance feature to periodically clean and flush debris using the bodies own fluids or a solution stored in the treatment system.

The electrical signals described above may be produced by an implanted generator or external stimulation device. The implanted generator may be powered and/or recharged from outside the body or may have its own power source.

The signals to the digestive system may be applied with leads and electrodes, or the electrodes could be part of a leadless generator(s) attached to parts of the digestive system. An external stimulation device may use magnetic induction coil or coils, or pads attached to the skin. Sensor data may be sent to the implanted generator via wires or wireless communication. Sensor data to an external device is sent by wireless telemetry.

The implanted generator system may have an external device for communication of settings to the generator and/or information from the generator to the external device. The external communication device and/or generator/stimulation device may store sensor data and/or stimulation signals and timing information. These devices may have a computer interface to download data to the computer for analysis and trending. Such data could also be used to modify the generator/stimulator programming to improve treatment.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method, comprising:
simultaneously monitoring at least one of nerve and muscle electrical activity on respective sides of a target plexus of a patient;
identifying desired electrical activity of at least one of nerves and muscles on both sides of the target plexus based on the monitored activity; and
modulating the electrical activity of the at least one of nerves and muscles on both sides of the target plexus to achieve a therapeutic result.

2. The method of claim 1, wherein the modulation is accomplished using at least one of (i) electrical current to one or more electrodes, and (ii) pharmaceuticals.

3. The method of claim 1, wherein the target plexus is one of the great plexuses.

4. The method of claim 3, wherein the target plexus is the celiac plexus.

5. The method of claim 3, wherein the target plexus is the hepatic plexus.

6. The method of claim 4, wherein the monitoring and modulation of the at least one of nerves and muscles are performed on nerves of the sympathetic or parasympathetic nervous system on one side of the celiac plexus and one or more vagus nerves on the other side of the celiac plexus.

7. The method of claim 6, further comprising: placing an electrode adjacent to or in communication with at least one ganglion along the sympathetic nerve chain and at least one of monitoring and modulating the at least one ganglion.

8. The method of claim 6, further comprising: placing an electrode adjacent to or in communication with the celiac plexus and at least one of monitoring and modulating the plexus.

9. The method of claim 1, wherein the modulation is accomplished using electrical current to the one or more electrodes and the method further includes: adjusting at least one parameter of one or more electrical signals to the electrodes until the physiological disorder has been demonstrably affected, modulated, treated, alleviated, arrested, or ameliorated.

10. The method of claim 1, wherein the step of modulating the electrical activity includes at least one of stimulating and reversibly blocking afferent or efferent signals of nervous and/or muscular tissue.

11. A method of treating obesity, comprising:
simultaneously monitoring electrical activity of at least two of: (i) at least one of the greater and lesser splanchnic nerves of the sympathetic nervous system, (ii) at least one of the gastric and celiac branches of the vagus nerve, and (iii) the celiac plexus, of a patient;
identifying desired electrical activity of at least one of the monitored nerves on both sides of the celiac plexus based on the monitored activity; and
modulating the electrical activity of at least one of the monitored nerves on both sides of the celiac plexus to achieve a therapeutic result.

12. The method of claim 11, further comprising: placing an electrode adjacent to or in communication with at least one ganglion along the sympathetic nerve chain and at least one of monitoring and modulating the at least one ganglion.

13. The method of claim 11, further comprising: placing an electrode adjacent to or in communication with the celiac plexus and at least one of monitoring and modulating the plexus.

14. The method of claim 11, further comprising:
placing one or more electrodes adjacent to or in communication with one or more nerves of the greater and lesser splanchnic nerves and at least one of monitoring and modulating such nerves; and
placing one or more electrodes adjacent to or in communication with one or more nerves of the vagus nerve and at least one of monitoring and modulating such nerves.

15. The method of claim 14, further comprising placing the one or more electrodes adjacent to or in communication with one or more nerves and/or muscles of a stomach of the patient.

16. The method of claim 14, further comprising placing the one or more electrodes adjacent to or in communication with one or more nerves and/or muscles associated with a sphincter of a cardiac orifice of the patient.

17. The method of claim 11, wherein the step of modulating the electrical activity includes at least one of stimulating and reversibly blocking afferent or efferent signals of nervous and/or muscular tissue.

18. A method of treatment, comprising:
simultaneously monitoring electrical activity of at least two of: (i) at least one nerve of the sympathetic nervous system, (ii) at least one nerve of the cranial nervous system, and (iii) at least one target plexus associated with the selected nerves of the sympathetic and cranial nervous systems, of a patient;
identifying desired electrical activity of at least one of the monitored nerves on both sides of the target plexus based on the monitored activity; and
modulating the electrical activity of at least one of the monitored nerves on both sides of the target plexus,
wherein the monitoring and modulating steps are directed to the treatment of one or more of: hyperhydrosis, pain syndromes, intestinal motility disorders, sexual dysfunction, liver disorders, pancreas disorders, heart disorders, pulmonary disorders, gastrointestinal disorders, and biliary disorders.

19. The method of treatment of claim 18, comprising further simultaneously monitoring electrical activity of at least one of: (i) muscles surrounding or interfacing with pathologically responding tissue, and (ii) any physical state of being that may be associated with the treatment.

20. The method of treatment of claim 18, further comprising creating a stimulation signal pattern based upon evaluation of the monitoring such that a desired therapeutic effect results.
